(19) United States
(12) Patent Application Publication
(10) Pub. No.: US 2020/0231926 A1
(43) Pub. Date: Jul. 23, 2020

(54) CELL CULTURING CONTROL METHOD, CELL CULTURING CONTROL DEVICE, CELL CULTURING DEVICE, AND CELL CULTURING SYSTEM

(71) Applicant: OLYMPUS CORPORATION, Tokyo (JP)

(72) Inventor: Ryosuke MURATA, Tokyo (JP)

(73) Assignee: OLYMPUS CORPORATION, Tokyo (JP)

(21) Appl. No.: 16/844,028

(22) Filed: Apr. 9, 2020

Related U.S. Application Data
(63) Continuation of application No. PCT/JP2018/047552, filed on Dec. 25, 2018.

(30) Foreign Application Priority Data
Dec. 28, 2017 (JP) 2017-253381

(51) Int. Cl. C12M 1/36; C12M 1/06; G06T 7/00

(52) U.S. Cl. CPC C12M 41/48; G06T 7/0012; C12M 27/02

(57) ABSTRACT

A cell culturing control method includes obtaining an image signal obtained by imaging at least part of an inside of a culture vessel, the culture vessel containing a liquid medium and floating cells that float in the medium; computing a cell feature value from the image signal, the cell feature value being related to sizes or shapes of the floating cells; and generating, on the basis of the cell feature value, a stirring control signal that controls stirring of the medium performed in the culture vessel.

### CROSS-REFERENCE TO RELATED APPLICATIONS

This is a continuation of International Application PCT/JP2018/047552 which is hereby incorporated by reference herein in its entirety. This application claims the benefit of Japanese Patent Application No. 2017-253381, the content of which is incorporated herein by reference.

### TECHNICAL FIELD

The present invention relates to a cell culturing control method, a cell culturing control device, a cell culturing device, and a cell culturing system.

### BACKGROUND ART

Recent years have seen the emergence of an industry for producing cells, such as iPS cells, using culture vessels such as bioreactors (for example, see PTL 1). Cells are cultured as they float in liquid media, and form colonies. The colony diameter increase with the progress of culturing. In PTL 1, an image of the inside of a culture vessel is obtained, the colony diameter distribution and colony count of the colonies in the medium are estimated from the image, and the quality and production of the cells are controlled on the basis of the estimated results.

### CITATION LIST

### Patent Literature

### {PTL 1} Japanese Unexamined Patent Application, Publication No. 2017-140006

### SUMMARY OF INVENTION

An aspect of the present invention provides a cell culturing control method that includes obtaining an image signal obtained by imaging at least part of an inside of a culture vessel, the culture vessel containing a liquid medium and floating cells that float in the medium; computing a cell feature value from the image signal, the cell feature value being related to sizes or shapes of the floating cells; and generating, on the basis of the cell feature value, a stirring control signal that controls stirring of the medium performed in the culture vessel.

Another aspect of the present invention provides a cell culturing control system that includes a cell culturing device; and a cell culturing control device connected to the cell culturing device via a communication network, wherein: the cell culturing device includes: a culture vessel that contains a liquid medium and floating cells that float in the medium; an imager that images at least part of an inside of the culture vessel to generate an image signal; and a stirring unit that stirs the medium in the culture vessel, and the cell culturing control device includes: a first processor that: obtains the image signal generated by the imager; computes a cell feature value from the image signal, the cell feature value being related to sizes or shapes of the floating cells; and generates, on the basis of the cell feature value, a stirring control signal that controls stirring of the medium performed in the culture vessel.

Yet another aspect of the present invention provides a non-transitory computer-readable medium having a program for controlling a cell culturing control system stored therein, the program causing a computer to execute functions of: obtaining an image signal obtained by imaging at least part of an inside of a culture vessel, the culture vessel containing a liquid medium and floating cells that float in the medium; computing a cell feature value from the image signal, the cell feature value being related to sizes or shapes of the floating cells; and generating, on the basis of the cell feature value, a stirring control signal that controls stirring of the medium performed in the culture vessel.

### BRIEF DESCRIPTION OF DRAWINGS

FIG. 1 is an overall structural diagram of a cell culturing system according to one embodiment of the present invention.

FIG. 2 is an overall structural diagram of a cell culturing device of the cell culturing system illustrated in FIG. 1.

FIG. 3 illustrates one example of an image signal generated by an imaging device of the cell culturing device illustrated in FIG. 2.

FIG. 4 illustrates one example of time-series image signals generated by the imaging device of the cell culturing device illustrated in FIG. 2.

FIG. 5 is a diagram illustrating one example of the results of the colony counting by a control server of the cell culturing system illustrated in FIG. 1.

FIG. 6 is a graph illustrating one example of a histogram generated by the control server of the cell culturing device illustrated in FIG. 1.

FIG. 7 is a flowchart illustrating a cell culturing control method using the cell culturing system illustrated in FIG. 1.

FIG. 8 is a continuation of the flowchart illustrated in FIG. 7.

FIG. 9 is an overall structural diagram of a modification of the cell culturing device illustrated in FIG. 2.

### DESCRIPTION OF EMBODIMENTS

A cell culturing system 100 according to one embodiment of the present invention will now be described with reference to the drawings.

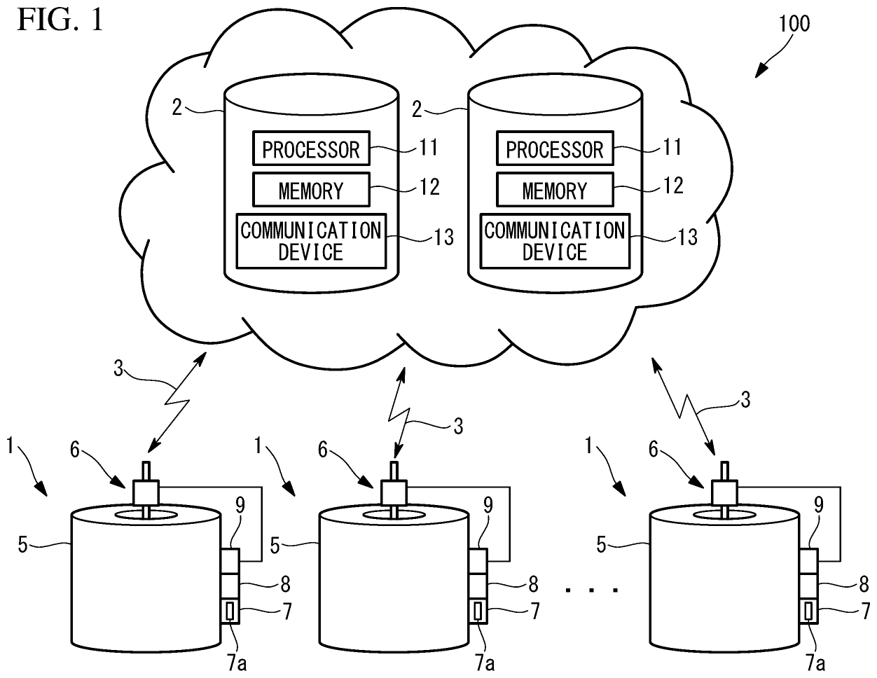

As illustrated in FIG. 1, the cell culturing system 100 of this embodiment includes cell culturing devices 1 that produce cells by culturing and growing cells, and control servers (cell culturing control devices) 2 that control the cell culturing devices 1.

The cell culturing devices 1 and the control servers 2 are connected to one another via a communication network 3. The communication network 3 is, for example, the Internet, an intranet, a local area network (LAN), a wide area network (WAN), or any combination of these. The communication network 3 may be wireless or wired.

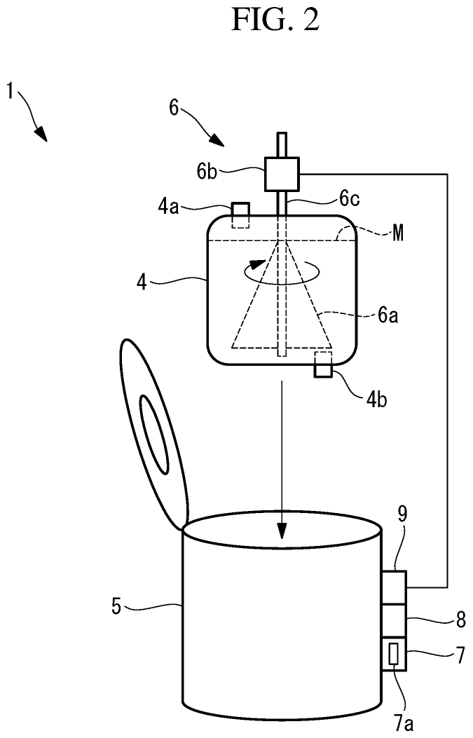

As illustrated in FIG. 2, each cell culturing device 1 is equipped with a culture vessel 4, a housing 5 that contains the culture vessel 4, a stirrer (stirring unit) 6 that stirs a medium M in the culture vessel 4, an imaging device 7 that images the inside of the culture vessel 4, a communication device (communication unit) 8 that communicates with the control servers 2 via the communication network 3, and a controller (stirring controller unit) 9 that controls the stirrer 6, the imaging device 7, and the communication device 8. The imaging device 7, the communication device 8, and the controller 9 are fixed to the housing 5.

The culture vessel 4 is a vessel for mass cell culturing, and is, for example, a bag or a cylindrical chamber used as a bioreactor. The culture vessel 4 contains a liquid medium M and cells that float in the medium M. At least part of the culture vessel 4 is transparent so that an image of the inside of the culture vessel 4 can be captured by the imaging device 7 disposed outside the culture vessel 4. The culture vessel 4 has a supply port 4a and a discharge port 4b. The medium M containing cells is supplied into the culture vessel 4 through the supply port 4a, and the medium M containing cells is discharged from the culture vessel 4 through the discharge port 4b. The cells are floating cells that grow and proliferate while floating in the medium M. In the medium M, the cells exist as single cells or in the form of substantially spherical colonies C.

The temperature, the humidity, the carbon dioxide concentration, etc., of the inside the housing 5 can be adjusted so that an environment appropriate for the cell culturing is maintained inside.

The stirrer 6 is equipped with a stirring blade 6a disposed inside the culture vessel 4, a stirring drive device 6b disposed outside the culture vessel 4, and a shaft 6c that connects the stirring blade 6a and the stirring drive device 6b. The shaft 6c is inserted into the inside of the culture vessel 4 from the outside while the hermetical seal of the culture vessel 4 is maintained. The stirring drive device 6b rotates the shaft 6c about the longitudinal axis of the shaft 6c so as to rotate the stirring blade 6a about the shaft 6c.

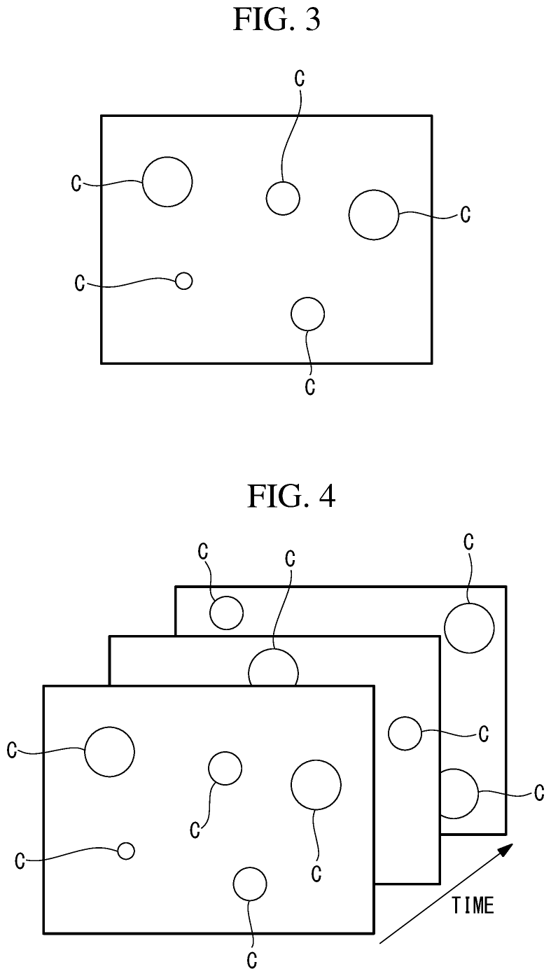

The imaging device 7 is equipped with an imager 7a. The imaging device 7 may be a common camera. The imaging range of the imager 7a is determined on the basis of the angle of view and the depth of field of the imaging device 7. The imaging range is in the inside of the culture vessel 4 in the housing 5. The imager 7a images the imaging range, and, as illustrated in FIG. 3, generates an image signal that includes images of single cells and colonies C.

In order to image a wide range or the entirety of the inside of the culture vessel 4, one imaging device 7 may be used to capture images from multiple positions as it moves relative to the culture vessel 4, or multiple imaging devices 7 installed at multiple positions may be used.

The communication device 8 receives the image signal from the imager 7a and transmits the image signal to the control server 2 via the communication network 3. The communication device 8 receives a stirring control signal from the control server 2 via the communication network 3, and transmits the stirring control signal to the controller 9.

The controller 9 causes the imaging device 7 to execute regular image capturing according to a preset imaging schedule, and causes the communication device 8 to transmit the image signal generated by the imager 7a to the control server 2. As illustrated in FIG. 4, the controller 9 may command the imager 7a to capture images multiple times, and may command the communication device 8 to transmit time-series image signals to the control server 2. The controller 9 controls the stirring drive device 6b of the stirrer 6 according to the stirring control signal received via the communication device 8 from the control server 2.

This controller 9 is constituted by a processor and a memory. The memory stores a program according to which the processor executes processes for controlling the stirrer 6, the imaging device 7, and the communication device 8.

At least part of the structure of the cell culturing device 1 may be disposable. The culture vessel 4, the stirring blade 6a, and the shaft 6c that directly contact the medium M are preferably disposable in order to reliably prevent contamination. The housing 5, the imaging device 7, and the communication device 8 are preferably repeatedly used.

The control server 2 is, for example, a cloud server. The cloud server is on the Internet, and provides cloud services to the cell culturing device 1 via the communication network 3. Alternatively, the control server 2 may be a computer at a desired location.

The control server 2 is equipped with a processor 11, a memory 12, and a communication device (communication unit) 13 that communicates with the cell culturing device 1 via the communication network 3. The memory 12 stores a cell feature value computing program and a stirring control signal-generating program.

The processor 11 receives image signals from the imaging device 7 of the cell culturing device 1 through communication between the communication devices 8 and 13.

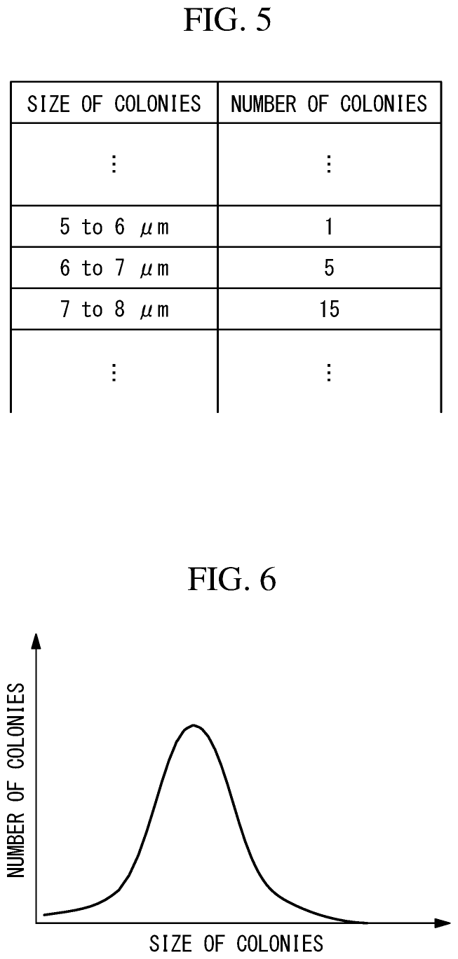

Next, the processor 11 executes the process of computing the cell feature value from the image signal according to the cell feature value computing program stored in the memory 12. Specifically, the processor 11 extracts the ring-shaped edges of colonies C from the image signal by edge extraction, and identifies regions surrounded by the edges as colonies C. Next, the processor 11 measures the size (diameter) of each colony C and counts the number of colonies C of respective sizes, as illustrated in FIG. 5. Next, the processor 11 generates a histogram of the sizes of the colonies C as illustrated in FIG. 6. In the histogram, the horizontal axis indicates the sizes of the colonies C, and the vertical axis indicates the number of colonies C.

Next, the processor 11 computes the cell feature value from the histogram. The cell feature value is a value that satisfies the following three conditions 1, 2, and 3.

Condition 1: a value obtained from the image signal Condition 2: a value related to the appearance of the cells

Condition 3: a value correlated with either the progress of cell culturing or the dispersion state of the cells in the medium

A preferable cell feature value that satisfies the above-described three conditions 1, 2, and 3 is the mode (the size at which the number of colonies C is the highest), the median, the mean, the variance, or the half-width of the histogram. As the culturing progresses, the colonies C grow in size as a whole, and variation in size among these colonies C widens. Thus, as the culturing progresses, the mode, median, mean, variance, and half-width increase.

When the processor 11 is to obtain multiple time-series image signals from the imaging device 7 of the cell culturing device 1, the processor 11 may compute one cell feature value from the multiple image signals.

The method for extracting cells from the image signal is not limited to edge extraction and may be any other method. For example, colonies C may be detected from the image signal through a neural network, such as deep learning.

Alternatively, depending on the cell type or the accuracy of image analysis, individual cells may be extracted from the image signal to detect the shapes of the respective cells and the number of cells.

Next, the processor 11 executes a process of generating a stirring control signal on the basis of the cell feature value according to the stirring control signal-generating program stored in the memory 12. The stirring control signal is a signal that indicates, to the stirrer 6, a parameter with which the state of stirring of the medium M can be controlled. The processor 11 estimates the progress of culturing on the basis of the cell feature value, sets a parameter appropriate for the progress, and generates a stirring control signal that causes the stirrer 6 to stir the medium M according to the set parameter.

The processor 11 transmits the stirring control signal to the cell culturing device 1 through communication between the communication devices 8 and 13.

For example, the parameter is the rotation rate (stirring rate) of the stirring blade 6a. The larger the mode, mean, or median of the sizes of the colonies C, the higher the set rotation rate. The rotation rate is determined by using a table stored in advance in the memory 12. The table indicates the cell feature values and the rotation rates associated with each other. The processor 11 reads out the table from the memory 12, refers to the table, selects the rotation rate corresponding to the computed cell feature value, and generates a stirring control signal that causes the stirring blade 6a to rotate at the selected rotation rate.

The control server 2 may be connected to one cell culturing device 1 via the communication network 3; alternatively, as illustrated in FIG. 1, the control server 2 may be connected to multiple cell culturing devices 1.

Alternatively, multiple control servers 2 that respectively correspond to the types of the cells to be cultured by the cell culturing devices 1 may be provided. For example, one control server 2 generates a stirring control signal optimized for culturing cells A, and another control server 2 generates a stirring control signal optimized for culturing cells B different from the cells A. In such a case, the cell culturing devices 1 communicate with the control servers 2 that correspond to the types of the cells, for example, and transmit image signals to and receive the stirring control signals from these control servers 2. The type of cells is designated by the user, for example.

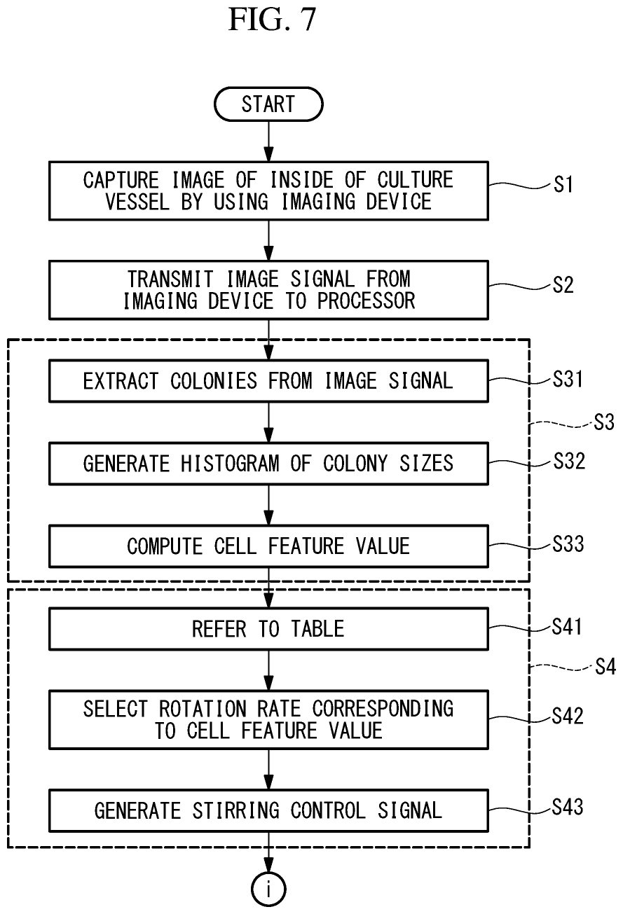

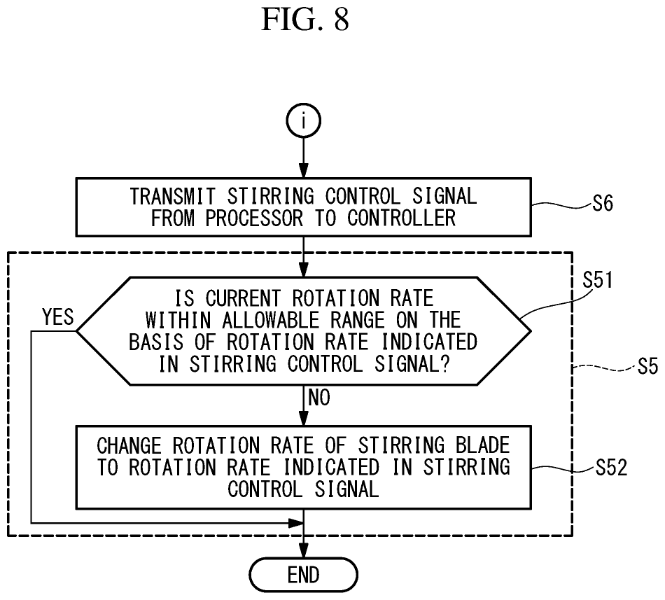

Next, a cell culturing control method that uses the cell culturing system 100 is described with reference to FIGS. 7 and 8.

In order to produce cells by using the cell culturing system 100, a medium M containing cells is supplied to the culture vessel 4 through the supply port 4a, the culture vessel 4 containing the medium M and the cells is placed in the housing 5, and the cells are cultured in an environment appropriate for culturing the cells inside the housing 5. During culturing, the medium M is stirred by the rotating stirring blade 6a disposed in the medium M so that the cells are evenly dispersed in the medium M. The cell culturing system 100 controls the stirring of the medium M during culturing according to the cell culturing control method illustrated in FIGS. 7 and 8.

The cell culturing control method according to an embodiment includes a step S1 of capturing an image of the inside of the culture vessel 4 with the imager 7a and generating an image signal; a step S2 of allowing the control server 2 to obtain the image signal; a step S3 of computing a cell feature value from the image signal; a step S4 of generating a stirring control signal on the basis of the cell feature value; and a step S5 of controlling the stirrer 6 according to the stirring control signal.

In step S1, the controller 9 causes the imager 7a to capture an image so as to generate an image signal of at least part of the culture vessel 4. The image signal is transmitted from the communication device 8 of the cell culturing device 1 to the communication device 13 of the control server 2 via the communication network 3.

Next, in step S2, the processor 11 receives the image signal from the communication device 13.

Next, in step S3, the processor 11 extracts the colonies C of the cells from the image signal (step S31), measures the size of each of the extracted colonies C, and generates a histogram of the sizes of the colonies C (step S32). Next, the processor 11 sets the mode, median, mean, variance, or half-width of the histogram as the cell feature value (step S33).

Next, in step S4, the processor 11 refers to the table stored in the memory 12 (step S41). The processor 11 selects the rotation rate corresponding to the cell feature value computed in step S3 (step S42), and generates a stirring control signal that causes the stirring blade 6a to rotate at the selected rotation rate (step S43).

The processor 11 transmits the stirring control signal to the controller 9 via the communication device 13, the communication network 3, and the communication device 8 (step S6).

Next, in step S5, the controller 9 controls the stirring drive device 6b according to the stirring control signal.

For example, the controller 9 judges whether the current rotation rate of the stirring blade 6a is within an allowable range (step S51). The allowable range of the rotation rate is the range determined on the basis of the rotation rate indicated in the stirring control signal. If the current rotation rate is within the allowable range (YES in step S51), the controller 9 maintains the current rotation rate. If the current rotation rate is outside the allowable range (NO in step S51), the controller 9 controls the stirring drive device 6b to change the rotation rate of the stirring blade 6a to the rotation rate indicated by the stirring control signal (step S52).

In order to produce homogeneous cells by using the cell culturing device 1, it is critical to keep a homogeneous environment in the culture vessel 4. Meanwhile, the colonies C of the cells move downward in the medium M due to gravitational force. Thus, the cells are cultured while stirring the medium M by the rotation of the stirring blade 6a in order to evenly disperse the colonies C of the cells in the medium M.

The colonies C grow in size with the progress of culturing. The cell dispersing effect achieved by the stirrer 6 differs depending on the sizes of the colonies C. For example, when the stirring rate of the medium M is constant, the effect of dispersing the colonies C would be degraded as the colonies C grow in size, and thus the density of the colonies C in the medium M becomes uneven. When a colony C becomes excessively large, differences may arise between the center portion and the peripheral portion of the colony C in terms of the distribution of nutrition, etc. Thus, the sizes of the colonies C is preferably kept to a particular size or smaller by disintegrating the colonies C by stirring the medium M.

Furthermore, in order to suppress the influence of stirring on the cells, the medium M stirring rate is preferably as low as possible.

In this embodiment, the image of the inside of the culture vessel 4 is regularly captured by the imaging device 7 during cell culturing. The cell feature value correlated with the progress of the culturing is computed from the image signal, a stirring control signal is generated on the basis of the cell feature value, and the stirring rate at which the stirrer 6 stirs the medium M is controlled according to the stirring control signal. In other words, the medium M stirring rate changes over time according to the changes in the sizes of the colonies C of the cells over time. In this manner, there is an advantage in that the medium M can be appropriately stirred according to the progress of the culturing, and homogeneous cells can be produced.

Furthermore, unlike the case in which a sensor or the like in contact with the liquid surface is used, an image signal that can be obtained without contacting the medium M is used to detect the progress of the culturing; thus, the culture vessel 4 can have a structure suitable for a disposable vessel.

In this embodiment, the rotation rate of the stirring blade 6a is controlled by the stirring control signal; alternatively or in addition to this, the shape of the stirring blade 6a may be controlled by the stirring control signal. In this case, the shape of the stirring blade 6a is changeable, and the stirring drive device 6b can change the shape of the stirring blade 6a.

The stirrer 6 may have multiple stirring blades 6a, and the stirring blades 6a may rotate at independent rotation rates. In such a case, the rotation rates of the stirring blades 6a can be controlled to be different from one another. For example, a stirring control signal may control a stirring blade 6a at a shallow position to rotate at a low rotation rate and a stirring blade 6a at a deep position to rotate at a high rotation rate.

The effect of dispersing the cells by stirring the medium M is affected by the temperature of the medium M and the amount of the medium M inside the culture vessel 4 in addition to the rotation rate (stirring rate). Thus, the processor 11 may obtain information regarding the temperature and amount of the medium M from the cell culturing device 1, and the rotation rate may be determined by considering the temperature and amount of the medium M in addition to the cell feature value. The temperature of the medium M is, for example, measured by a non-contact thermometer disposed outside the culture vessel 4. The amount of the medium M is, for example, input to the cell culturing device 1 by the user.

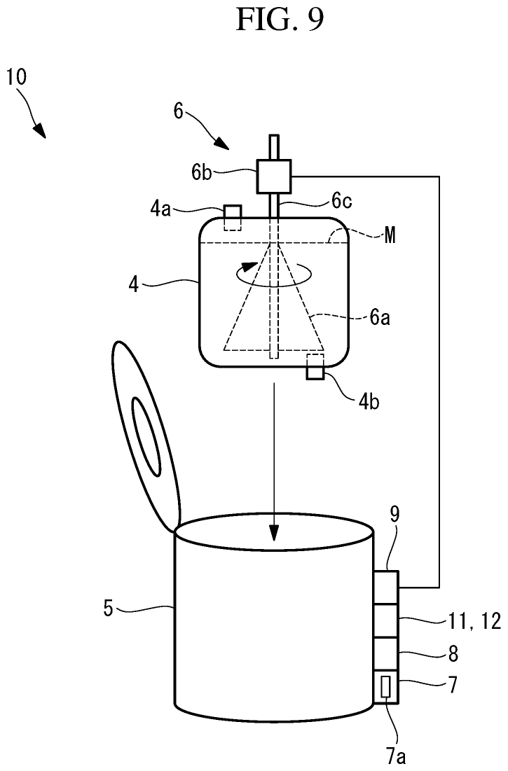

In this embodiment, the processor 11 and the memory 12 are installed in the control server 2 separate from the cell culturing device 1, and the computing of the cell feature value and generation of the stirring control signal are executed in the control server 2; alternatively, as illustrated in FIG. 9, the processor 11 and the memory 12 may be installed in a cell culturing device 10. That is, the cell culturing device 10 may also be a cell culturing control device. The processor 11 and the memory 12 may be separately provided from the controller 9, or may be the processor and the memory of the controller 9.

The cell culturing device 10 illustrated in FIG. 9 may transmit processing results related to the computing of the cell feature value and the processing results related to generation of the stirring control signal to the server via the communication network 3. The server may be the control server 2 or a different server.

The server receives the processing results from each of the multiple cell culturing devices 10, and the received processing results are stored in the memory 12 so that the cell feature values in previous culturing and the stirring control signals are stored in association with each other.

In step S4, the processor 11 obtains the cell feature values and the stirring control signals stored up to that time from the server prior to referring the table (step S41). Next, the processor 11 updates the table on the basis of the obtained cell feature values and the stirring control signals.

As described above, since multiple cell culturing devices 10 share the cell feature values and the stirring control signals, the table can be efficiently updated.

An evaluation of the quality of the produced cells may be input to the processor 11 by the user, and the evaluation may be transmitted to the server in association with the cell feature value and the stirring control signal of the culturing of these cells. The server receives and stores the processing results and the evaluation. The processor 11 of the cell culturing device 10 may receive the evaluation together with the cell feature value and the stirring control signal, and may update the table by using the cell feature value and the stirring control signal of the culturing for which a high evaluation is obtained.

In this embodiment, the value correlated with the progress of the cell culturing is used as the cell feature value; alternatively, a value correlated with the dispersion state of the cells in the medium may be used.

The cell feature value correlated with the dispersion state of the cells in the medium is, for example, the variance or the half-width of the histogram.

In this embodiment, the stirrer 6 that stirs the medium M by rotating the stirring blade 6a is used; alternatively, the stirrer of a different type may be used. For example, a stirrer may be a stirrer that circulates the medium M, a stirrer that causes a magnetic stirrer to rotate, or a stirrer that causes the culture vessel 4 to shake.

In the embodiments and modifications described above, the structure in which the imaging device 7 is fixed to the housing 5 of the cell culturing device 1 or 10 is described; alternatively, the imaging device 7 may be detachably attachable to the culture vessel 4.

The above-described embodiment also leads to the following aspects.

An aspect of the present invention provides a cell culturing control method that includes a step of obtaining an image signal obtained by imaging at least part of an inside of a culture vessel, the culture vessel containing a liquid medium and floating cells that float in the medium; a step of computing a cell feature value from the image signal, the cell feature value being correlated with either progress of culturing of the floating cells or a dispersion state of the floating cells in the medium; and a step of generating, on the basis of the cell feature value, a stirring control signal that controls stirring of the medium in the culture vessel.

According to this aspect, the image signal of the floating cells cultured in the culture vessel is obtained, the cell feature value is computed from the image signal, and the stirring control signal is generated on the basis of the cell feature value. The cell feature value is a value correlated with the progress of the culturing of the floating cells or the dispersion state of the floating cells in the medium. Thus, the medium can be stirred, in a manner appropriate for the progress of the culturing, according to the stirring control signal based on the cell feature value.

In the aspect described above, the method may further include a step of capturing an image of the at least part of the inside of the culture vessel by using an imager to generate the image signal, in which the step of obtaining an image signal includes a step of receiving the image signal from the imager by communicating with the imager.

In the aspect described above, the step of computing a cell feature value may include a step of extracting colonies of the floating cells from the image signal; a step of generating a histogram of sizes of the colonies; and a step of setting, as the cell feature value, any one of a mode, a median, a mean, a variance, and a half-width of the sizes in the histogram.

The floating cells that are cultured while floating in the medium form substantially spherical colonies, and the colonies grow in size (diameter) as the culturing progresses. In other words, as the culturing progresses, the mode, the median, the mean, the variance, and the half-width of the colonies increase. Thus, by using these values as the cell feature value, the progress of the culturing of the floating cells can be accurately determined.

In the aspect described above, the step of generating a stirring control signal may include a step of referring to a table in which cell feature values and stirring rates of the medium are associated with each other; and a step of selecting, from the table, the stirring rate corresponding to the cell feature value computed in the step of computing the cell feature value, and generating the stirring control signal that causes stirring of the medium at the selected stirring rate.

In this manner, the stirring rate of the medium can be controlled on the basis of the cell feature value.

In the aspect described above, the step of generating the stirring control signal may include a step of updating the table by using a correspondence relationship between the cell feature value computed in previous culturing of floating cells and the stirring control signal.

In this manner, the table can be updated on the basis of the culturing carried out in the past so that the correspondence relationship between the cell feature value and the stirring rate is more appropriate.

In the aspect described above, the step of generating a stirring control signal may include a step of obtaining a temperature of the medium in the culture vessel; and a step of generating the stirring control signal on the basis of both the cell feature value and the temperature.

The optimum stirring conditions for the medium depend on the temperature of the medium also. Thus, a stirring control signal that can more appropriately stir the medium can be generated by considering not only the cell feature value but also the temperature of the medium.

Another aspect of the present invention provides a cell culturing control device that includes a processor, in which the processor obtains an image signal obtained by imaging at least part of an inside of a culture vessel, the culture vessel containing a liquid medium and floating cells that float in the medium; computes a cell feature value from the image signal, the cell feature value being correlated with either progress of culturing the floating cells or a dispersion state of the floating cells in the medium; and generates, on the basis of the cell feature value, a stirring control signal that controls stirring of the medium in the culture vessel.

According to this aspect, the processor obtains the image signal of the floating cells cultured in the culture vessel, computes the cell feature value from the image signal, and generates a stirring control signal on the basis of the cell feature value. The cell feature value is a value correlated with the progress of the culturing of the floating cells or the dispersion state of the floating cells in the medium. Thus, the medium can be stirred, in a manner appropriate for the progress of the culturing, according to the stirring control signal based on the cell feature value.

In the aspect described above, the cell culturing control device may further include a culture vessel containing the medium and the floating cells; an imager that images the at least part of the inside of the culture vessel and generates the image signal; a stirring unit that stirs the medium in the culture vessel; and a stirring control unit that controls the stirring unit according to the stirring control signal generated by the processor.

As described above, since the cell culturing control device is equipped with components of a cell culturing device, namely, the culture vessel, the imager, the stirring unit, and the stirring control unit, the cell culturing control device can also serve as a cell culturing device.

In the aspect described above, the cell culturing control device may further include a communication unit that transmits at least one of a processing result related to the computing of the cell feature value and a processing result related to generation of the stirring control signal to a server via a communication network.

In this manner, the information related to the cell feature value and the stirring control signal can be stored in the server, and the stored information can be shared among multiple cell culturing control devices connected to the server.

In the aspect described above, the cell culturing control device may further include a communication unit, in which the communication unit is connected, via a communication network, to a cell culturing device equipped with the culture vessel containing the medium and the floating cells, an imager that images at least part of an inside of the culture vessel, a stirring unit that stirs the medium in the culture vessel, and a stirring control unit that controls the stirring unit according to the stirring control signal generated by the processor, and the communication unit receives the image signal from the imager via the communication network and transmits the stirring control signal generated by the processor to the stirring control unit via the communication network.

When this communication unit is provided, the operation of the stirring unit can be remotely controlled by the stirring control unit of the cell culturing device.

Yet another aspect of the present invention provides a cell culturing device that cultures cells by using a culture vessel that contains a liquid medium and floating cells that float in the medium, the device including: an imager that images at least part of an inside of the culture vessel and generates an image signal; a stirring unit that stirs the medium in the culture vessel; a communication unit that transmits the image signal to a cell culturing control device via a communication network and receives a stirring control signal from the cell culturing control device via the communication network, the stirring control signal being generated on the basis of a cell feature value computed from the image signal and correlated with either progress of culturing the floating cells or a dispersion state of the floating cells in the medium; and a stirring control unit that controls the stirring unit according to the stirring control signal received by the communication unit.

According to this aspect, the image signal of the floating cells cultured in the culture vessel is generated by the imager, the image signal is transmitted to the cell culturing control device by the communication unit, the stirring control signal based on the image signal from the cell culturing control device is received by the communication unit, and the stirring control unit causes the stirring unit to operate according to the stirring control signal.

In this case, the stirring control signal generated in the cell culturing control device is a signal based on the cell feature value correlated with the progress of the culturing of the floating cells or the dispersion state of the floating cells in the medium. Thus, the medium can be stirred, in a manner appropriate for the progress of the culturing, according to the stirring control signal.

Still another aspect of the present invention provides a cell culturing device that cultures cells by using a culture vessel that contains a liquid medium and floating cells that float in the medium, the device including: an imager that images at least part of an inside of the culture vessel and generates an image signal; a stirring unit that stirs the medium in the culture vessel; a communication unit that transmits the image signal to a cell culturing control device via a communication network and receives a stirring control signal from the cell culturing control device via the communication network, the image signal being used to compute a cell feature value correlated with either progress of culturing the floating cells or a dispersion state of the floating cells in the medium and to generate the stirring control signal on the basis of the cell feature value; and a stirring control unit that controls the stirring unit according to the stirring control signal received by the communication unit.

According to this aspect, the image signal of the floating cells cultured in the culture vessel is generated by the imager, the image signal is transmitted to the cell culturing control device by the communication unit, the stirring control signal based on the image signal from the cell culturing control device is received by the communication unit, and the stirring control unit causes the stirring unit to operate according to the stirring control signal.

In this case, the image signal transmitted to the cell culturing control device is used to compute a cell feature value correlated with the progress of the culturing of the floating cells or the dispersion state of the floating cells in the medium. Thus, the medium can be stirred, in a manner appropriate for the progress of the culturing, according to the stirring control signal based on the cell feature value.

Yet another aspect of the present invention provides a cell culturing system including: a cell culturing device that cultures cells by using a culture vessel that contains a liquid medium and floating cells that float in the medium; and a cell culturing control device connected to the cell culturing device via a communication network. The cell culturing device includes: an imager that images at least part of an inside of the culture vessel and generates an image signal; a stirring unit that stirs the medium in the culture vessel; and a stirring control unit that controls the stirring unit according to a stirring control signal received from the cell culturing control device. The cell culturing control device includes a processor that computes, from the image signal, a cell feature value correlated with progress of culturing the floating cells or a dispersion state of the floating cells in the medium, and generates, on the basis of the cell feature value, the stirring control signal that controls stirring of the medium in the culture vessel.

According to this aspect, the image signal generated by the imager is transmitted from the cell culturing device to the cell culturing control device via the communication network, and the stirring control signal generated by the processor is transmitted from the cell culturing control device to the cell culturing device via the communication network. In the cell culturing device, the stirring control unit causes the stirring unit to operate according to the stirring control signal.

In this case, the stirring control signal generated in the cell culturing control device is a signal based on the cell feature value correlated with the progress of the culturing of the floating cells or the dispersion state of the floating cells in the medium in the cell culturing device. Thus, the medium can be stirred, in a manner appropriate for the progress of the culturing, according to the stirring control signal.

### REFERENCE SIGNS LIST

100 cell culturing system
1 cell culturing device
10 cell culturing device (cell culturing control device)
2 control server (cell culturing control device)
3 communication network
4 culture vessel
5 housing
6 stirrer (stirring unit)
7 imaging device
7a imager
8 communication device (communication unit)
9 controller (stirring control unit)
11 processor
12 memory
13 communication device (communication unit)
M medium
C colony

1. A cell culturing control method comprising:
obtaining an image signal obtained by imaging at least part of an inside of a culture vessel, the culture vessel containing a liquid medium and floating cells that float in the medium;
computing a cell feature value from the image signal, the cell feature value being related to sizes or shapes of the floating cells; and
generating, on the basis of the cell feature value, a stirring control signal that controls stirring of the medium performed in the culture vessel.

2. The cell culturing control method according to claim 1, wherein the cell feature value related to sizes of colonies is computed in the computing of the cell feature value.

3. The cell culturing control method according to claim 1, wherein the computing of the cell feature value includes:
extracting the floating cells from the image signal;
generating a histogram of sizes of the floating cells; and
setting, as the cell feature value, any one of a mode, a median, a mean, a variance, and a half-width of the sizes in the histogram.

4. The cell culturing control method according to claim 3, wherein the floating cells extracted from the image signal are colonies.

5. The cell culturing control method according to claim 4, further comprising:
imaging the at least part of the inside of the culture vessel by using an imager to generate the image signal,
wherein the obtaining of the image signal includes receiving the image signal from the imager by communicating with the imager.

6. The cell culturing control method according to claim 5, wherein the generating of the stirring control signal includes:
referring to a table in which the cell feature value and stirring rate of the medium are associated with each other; and
selecting, from the table, the stirring rate corresponding to the computed cell feature value, and generating the stirring control signal that causes stirring of the medium at the selected stirring rate.

7. The cell culturing control method according to claim 6, wherein the generating of the stirring control signal includes:
updating the table by using a corresponding relationship between the cell feature value computed in previous culturing of floating cells and the stirring control signal.

8. A cell culturing control system comprising:
a cell culturing device; and
a cell culturing control device connected to the cell culturing device via a communication network, wherein:
the cell culturing device includes:
a culture vessel that contains a liquid medium and floating cells that float in the medium;
an imager that images at least part of an inside of the culture vessel to generate an image signal; and
a stirring unit that stirs the medium in the culture vessel, and
the cell culturing control device includes:
a first processor that:
obtains the image signal generated by the imager;
computes a cell feature value from the image signal, the cell feature value being related to sizes or shapes of the floating cells; and
generates, on the basis of the cell feature value, a stirring control signal that controls stirring of the medium performed in the culture vessel.

9. The cell culturing control system according to claim 8, wherein the cell feature value is a cell feature value related to sizes of colonies.

10. The cell culturing control system according to claim 8, wherein the cell feature value includes any one of a mode, a median, a mean, a variance, and a half-width of sizes of the floating cells.

11. The cell culturing control system according to claim 10, wherein the floating cells extracted from the image signal are colonies.

12. The cell culturing control system according to claim 11, wherein the stirring control signal includes a command for controlling a rotation rate of the stirring unit.

13. The cell culturing control system according to claim 12, wherein
the cell culturing device further includes a controller including a second processor,
the controller receives the stirring control signal generated by the first processor, and
the second processor controls the stirring unit on the basis of the stirring control signal.

14. The cell culturing control system according to claim 13, wherein the cell culturing control device further includes a communication unit that receives the image signal from the imager via the communication network and transmits the stirring control signal generated by the first processor to the second processor via the communication network.

15. The cell culturing control system according to claim 12, wherein
the stirring unit includes multiple stirring blades, and
the stirring control signal includes a command for rotating the multiple stirring blades at independent ratation rates.

16. The cell culturing control system according to claim 8, wherein
the cell culturing device and the cell culturing control device are integrally formed, and
the first processor controls the stirring unit on the basis of the stirring control signal.

17. A non-transitory computer-readable medium having a program for controlling a cell culturing control system stored therein, the program causing a computer to execute functions of:
obtaining an image signal obtained by imaging at least part of an inside of a culture vessel, the culture vessel containing a liquid medium and floating cells that float in the medium;
computing a cell feature value from the image signal, the cell feature value being related to sizes or shapes of the floating cells; and
generating, on the basis of the cell feature value, a stirring control signal that controls stirring of the medium performed in the culture vessel.

18. The medium according to claim 17, wherein the cell feature value related to sizes of colonies is computed in the computing of the cell feature value.
